(19) United States
(12) Patent Application Publication
(10) Pub. No.: US 2019/0000589 A1
(43) Pub. Date: Jan. 3, 2019

(54) Surgical Article And Method For Managing Surgical Articles During A Surgical Procedure

(71) Applicant: STRYKER CORPORATION, Kalamazoo, MI (US)

(72) Inventors: Brian James VANDERWOUDE, Portage, MI (US); Michael Allan ROUX, Trabuco Canyon, CA (US)

(73) Assignee: Stryker Corporation, Kalamazoo, MI (US)

(21) Appl. No.: 16/064,560

(22) PCT Filed: Oct. 14, 2016
(86) PCT No.: PCT/US2016/057077

§ 371 Date: Jun. 21, 2018

Related U.S. Application Data
(60) Provisional application No. 62/270,405, filed on Dec. 21, 2015.

(51) Int. Cl. A61B 90/96; A61F 13/44; A61F 13/551

(52) U.S. Cl. CPC A61B 90/96; A61B 2090/0805; A61F 13/551; A61F 13/44

(57) ABSTRACT

An exemplary method for managing surgical sponges in an operating room during a surgical procedure is provided. The method includes providing a first surgical sponge and a second surgical sponge. The first surgical sponge includes a first optically-scannable element, a first human-readable element, and a first detecting element, which includes a first set of unique identification information. The second surgical sponge includes a second optically-scannable element, a second human-readable element, and a second detecting element, which include a second set of unique identification information. The first and second sets of unique identification information are different from one another. The method further includes counting-in the first and second surgical sponges by optically-scanning.

### CROSS REFERENCE TO RELATED APPLICATIONS

This application claims the benefit of U.S. Provisional Patent Application No. 62/270,405, filed Dec. 21, 2015, which is hereby incorporated by reference in its entirety.

### TECHNICAL FIELD

The present invention relates to a method for counting and detecting the a surgical article having multiple counting and detecting elements

### BACKGROUND OF THE DISCLOSURE

Surgical sponges are comprised of absorbent material for soaking up blood and other bodily fluids in and around an incision site. Health care professionals (HCPs) typically follow strict procedures to account for each and every sponge used during a surgery, in view of the risks associated with a surgical sponge being inadvertently retained inside a patient. In addition, handling and exposure to soiled sponges should be kept to a minimum because they can be a source of contamination. Thus, it is desirable to efficiently manage surgical articles in an operating room to reduce or eliminate the risks associated with surgical articles being retained inside a patient and the risks associated with the transmission of bloodborne diseases.

### SUMMARY OF THE DISCLOSURE

A method for managing surgical sponges in an operating room during a surgical procedure is provided. The method includes the step of providing a first surgical sponge and a second surgical sponge. The first surgical sponge includes a first optically-scannable element, a first human-readable element, and a first detecting element. Both of the first optically-scannable element and the first human-readable element include a first set of unique identification information. The second surgical sponge includes a second optically-scannable element, a second human-readable element, and a second detecting element. Both of the second optically-scannable element and the second human-readable element include a second set of unique identification information. The first set of unique identification information is different from the second set of unique identification information. The method further includes the step of counting-in the first and second surgical sponges by optically-scanning. The first surgical sponge is collected and counted-out. A location of the second surgical sponge is detected by using the second detecting element after collecting the first surgical sponge. The second surgical sponge is collected after its location has been detected, and the second surgical sponge is counted-out.

### BRIEF DESCRIPTION OF THE DRAWINGS

Referring now to the drawings, exemplary illustrations are shown in detail. Although the drawings represent schematic embodiments, the drawings are not necessarily to scale and certain features may be exaggerated to better illustrate and explain an innovative aspect of an illustrative embodiment. Further, the exemplary illustrations described herein are not intended to be exhaustive or otherwise limiting or restricting to the precise form and configuration shown in the drawings and disclosed in the following detailed description. Exemplary illustrations are described in detail by referring to the drawings as follows:

FIG. 1A is a perspective view of one embodiment of a pack of surgical articles in the form of a stack of folded surgical sponges having counting elements and detecting elements configured to be counted and detected during a surgical procedure;

FIG. 1B is an enlarged plan view of the pack of FIG. 1A, as taken from encircled portion 1B, illustrating the pack having counting elements in the form of a master optically-scannable element and a master human readable element printed on a master tag;

FIG. 2A is a perspective view of another embodiment of a pack of surgical articles in the form of surgical instruments;

FIG. 2B is a perspective view of still another embodiment of a pack of surgical articles in the form of surgical sutures;

FIG. 3A is a plan view of one embodiment of first and second surgical sponges of the pack of FIG. 1A, illustrating each of the first and second surgical sponges having only one tag carrying the counting elements and a detecting element;

FIG. 3B is an enlarged view of the first surgical sponge of FIG. 3A, as taken from the encircled portion 3B, illustrating the first surgical sponge having counting elements in the form of a first optically-scannable element and a first human-readable element;

FIG. 3C is an enlarged view of the second surgical sponge of FIG. 3A, as taken from the encircled portion 3C, illustrating the second surgical sponge having counting elements in the form of a second optically-scannable element and a second human-readable element;

FIG. 4A is a plan view of another embodiment of first and second surgical sponges of the pack of FIG. 1A, illustrating the sponges having a detectable element configured to be detectable within a patient's body;

FIG. 4B is a plan view of another embodiment of first and second surgical sponges of the pack of FIG. 1A, illustrating the sponges having a detectable element configured to be detectable within an operating room but not within the patient's body;

FIG. 4C is a plan view of another embodiment of first and second surgical sponges of the pack of FIG. 1A, illustrating each of the first and second surgical sponges having two tags positioned on opposite corners and carrying the counting elements and the detecting elements;

FIG. 5 is a top schematic view of an operating room, illustrating the surgical articles of FIG. 4A detectable within a body of a patient and the surgical articles of FIG. 4B detectable within the operating room but not within the body of the patient; and

FIG. 6 is a flowchart of one embodiment of a method for counting and detecting surgical articles used during a surgical procedure.

### DETAILED DESCRIPTION OF THE DISCLOSURE

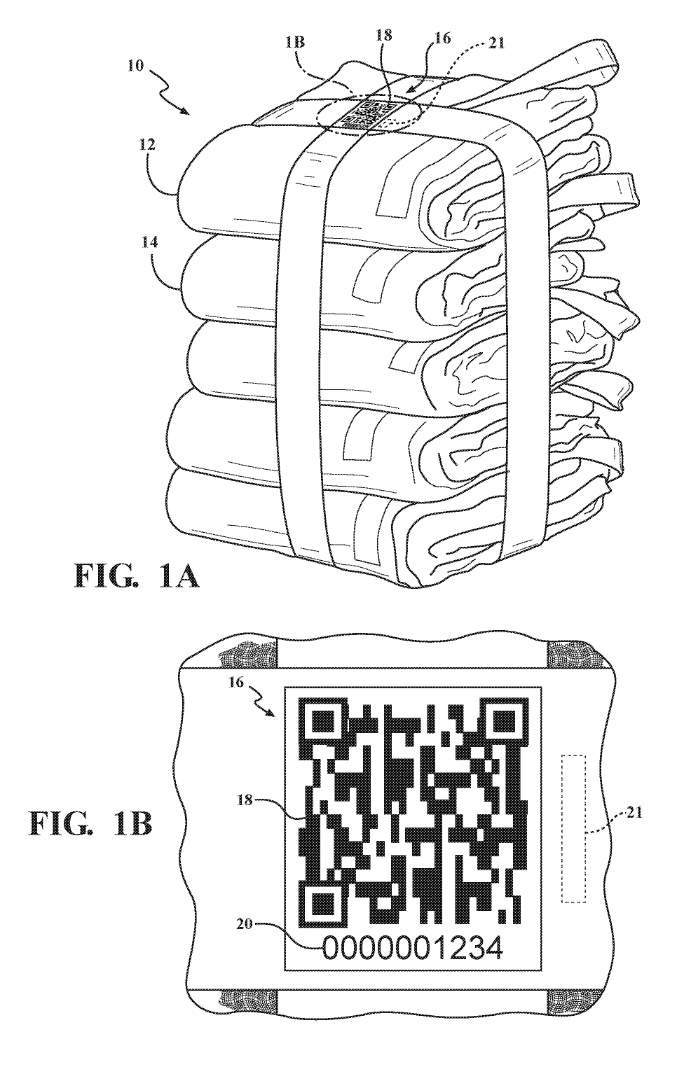

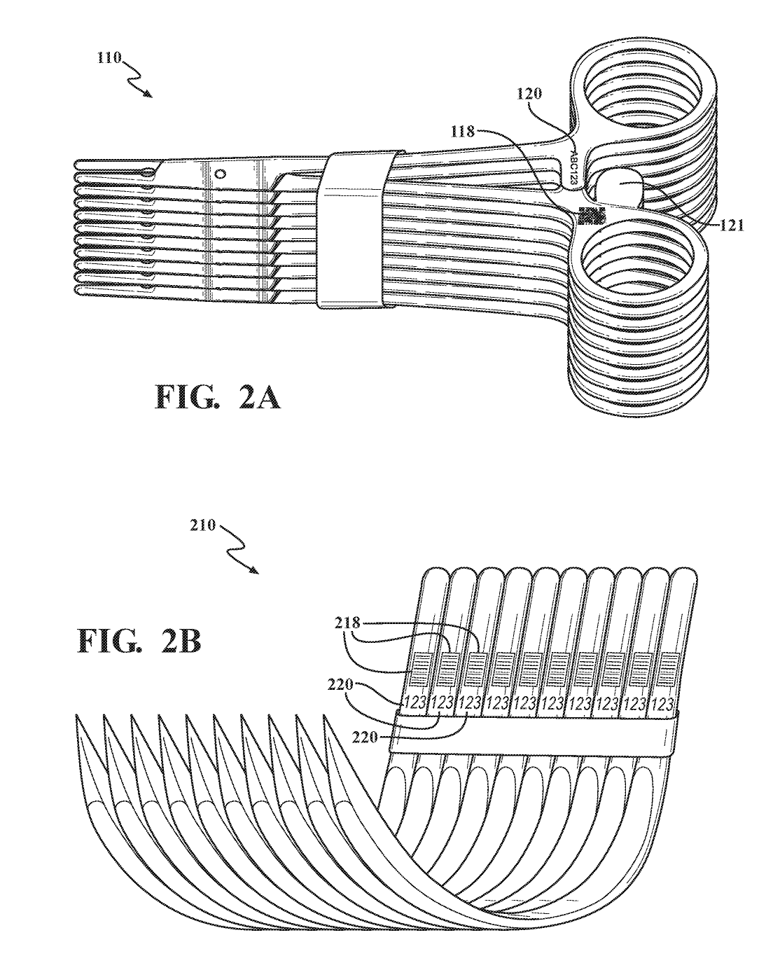

The present disclosure relates to a surgical article having one or more counting elements, one or more detecting elements, or any combination thereof, for counting and/or detecting the surgical article before, during, or after a surgical procedure. Non-limiting embodiments of the surgical article can include sponges, laparotomy pads, gauzes, implants, towels, suture needles, clips, staples, or surgical instruments. In particular, an embodiment of surgical articles illustrated in FIG. 1A can comprise a pack of sponges 10. Another embodiment of the pack of surgical articles can comprise a pack of surgical instruments 110 (FIG. 2A), which is similar to the pack of sponges 10 of FIG. 1A and, as described in detail below, includes similar components identified by the same reference numbers increased by 100. Still another embodiment of the pack of surgical articles can comprise a pack of surgical sutures 210 (FIG. 2B), which is similar to the pack of sponges 10 of FIG. 1A and, as described in detail below, includes similar components identified by the same reference numbers increased by 200. The counting element(s), the detecting element(s), or any combination thereof may be incorporated within tags, handles, or other portions of the surgical article. As described in detail below, each surgical article can include one or more tags, and each tag may include various combinations of the counting elements and the detecting elements. Another embodiment of the surgical article may include two, three, four or more tags. One of these tags can include one detecting element, such as a RFID element, and two counting elements, such as an optically-scannable element and a human-readable element. However, each tag can include any number of counting elements and any number of detecting elements.

The counting element may be configured to include unique identification information for each surgical article. The unique identification information may comprise a serial number or other identifier that is unique and assigned only to the corresponding article. In other embodiments, the unique identification information may further convey the type, size, weight, manufacturing dates, expiration date, number of similar articles in a corresponding pack, and/or other information used for counting or detecting the article.

The counting element may convey unique identification information by achieving one or more of the following functions: 1) being optically-scannable; 2) being human-readable; 3) transmitting an electromagnetic signal or wave corresponding with the unique identification information; and 4) being machine-readable in some other manner by conventional reading and/or scanning devices. Each surgical article may include one counting element scannable by an optical-scanning device or manually entered into a user interface sub-assembly of the scanning device, computer, or other system. However, each surgical article can include two, three, four, or more counting elements capable of providing these functions for the associated surgical article to count the surgical article by using one or more backup or redundant counting elements when, for example, another counting element is cut, sullied, or otherwise rendered inoperable. The counting elements on each surgical article may be different from one another, yet include the same unique identification information.

The scanning device may be configured to maintain a record of the surgical articles used in the procedure in on-board memory, or in cooperation with a server. At the conclusion of the surgical procedure, the records can be transmitted to a server and matched with patient records, such as electronic medical records, to update the same and provide an indication of which specific surgical articles were used with each patient at which times.

Each one of the counting elements may be encapsulated in biocompatible plastic coating or housing that is water-impermeable and sterilizable. Each counting element may be rigid to increase its service life. In other embodiments, the counting element can be flexible to permit the surgical article and the counting element to be folded or otherwise shaped in a more preferable manner for use within a patient's body.

To be optically-scannable, the surgical article may have one or more counting elements in the form of optically-scannable elements incorporated within one or more tags, handles, or other portions of the surgical article. The optically-scannable element may be scanned by an optical-scanning device to provide or determine the unique identification information associated with the article. Examples of the optically-scannable element can include a bar code, a QRS code, a data matrix, a symbol, or any combination thereof incorporated into the tags, handles, or other portions of the article in any number of ways. As but one example, the optically-scannable element can be a bar code printed on one or more tags of a surgical article.

In certain embodiments, each surgical article may include two or more redundant optically-scannable elements. These optically-scannable elements may be coupled to two or more tags, handles, or other portions of the surgical article that are spaced apart from one another. This allows the optical-scanning device to count the surgical article when, for example, one of the optically-scannable elements is cut, sullied or otherwise rendered unscannable. While the optically-scannable elements may be scanned by optical-scanning devices to provide or determine unique identification information, the optically-scannable elements may be configured to be scannable by mutually exclusive optical-scanning devices and/or technologies. For example, a single article may include two redundant optically-scannable elements including a QRS code and a bar code. Thus, a backup or secondary optical-scanning device may be used to scan a backup optically-scannable element, such as the bar code, when a primary optical-scanning device is inoperable and cannot be used to scan a primary optically-scannable element, such as the QRS code.

Each one of the optically-scannable elements may be encapsulated in biocompatible plastic coating or housing that is water-impermeable and sterilizable. Each optically-scannable element may be rigid to increase its service life. In other embodiments, the optically-scannable element can be flexible to permit the surgical article and the optically-scannable element to be folded or otherwise shaped in a more preferable manner for use within a patient's body.

In this embodiment, the optical-scanning device may also be configured to maintain the record of surgical articles used in the procedure in on-board memory, or in cooperation with the server through a remote database. However, it is contemplated other suitable scanning devices can be configured to maintain the record of surgical articles used in the procedure. At the conclusion of the surgical procedure, the records can be transmitted to the server and matched with patient records, such as electronic medical records, to update the same and provide an indication of which specific surgical articles were used with each patient at which times.

The optical-scanning device may include a scanning head configured to identify the corresponding optically-scannable element. For example, if the optically-scannable elements are bar codes, the associated scanning head is a device capable of optically scanning the bar codes. Some scanners include a camera head, which can be a transducer able to generate electronic signals that represent an image of at least a portion of the article being scanned. The optical-scanning device may also include user interface sub-assemblies having one or more input devices for manually inputting information, and one or more output devices for communicating information. It should be understood that to enable input, examples of the input devices may comprise one or more of: a touch screen display; physical buttons; a keyboard; a microphone; any other suitable input device; or any combination thereof. Furthermore, it should be understood that to communicate information, examples of the output devices may include one or more of: a touch screen display; a standard LCD display; a light; an audio output device; a vibratory motor; other haptic response mechanisms; or any combination thereof. The optical-scanning device may include a processor, and the data entered into the input device is electronically transferred to the processor. The processor generates the information that is communicated using the designated output devices. The optical-scanning device may also include a transmitter/receiver in certain embodiments, and the transmitter/receiver wirelessly exchanges signals with a transmitter/receiver to which a central server is connected.

To be human-readable, the surgical article may comprise one or more counting elements in the form of human-readable elements, which are readable or comprehensible by a health care professional (HCP) without the aid of a computer/processor, and the human readable elements may be incorporated within one or more tags, handles, or other portions of the surgical article. The HCP may read the human-readable element and then operate the input device of the optical-scanning device, a computer, or other system to provide, determine, or match the corresponding unique identification information of the surgical article based on the human-readable element. In one embodiment, the HCP manually enters the human-readable element into the input device, such as a keyboard, and the optical-scanning device determines the unique identifying information based on the human-readable element. In another embodiment, the HCP may use the input device to select a matching human-readable element, which had been previously stored in the optically-scanning device, which in turn provides the unique identification information corresponding with the previously stored human-readable element. The human-readable element may include the same unique identification information provided by the optically-scannable element. In certain embodiments of the surgical article that have both the human-readable element and one or more optically-scannable elements, the human-readable element can be utilized as a backup or secondary device for determining the unique identification information when the optically-scannable element is cut, sullied, or otherwise rendered inoperable. Embodiments of the human-readable element can include one or more of: an alphanumeric identifier; a symbol; or any type of visual or tactile indicator that is capable of being recognized by the HCP without the use of a machine. In other words, the human-readable element allows the HCP to determine unique identification information for each article without the use of any machine, such as the optical-scanning device. Other non-limiting embodiments of the human-readable element can include various indentations, patterns, colors, labels or combinations thereof. The human-readable element may be incorporated into the tags, handles, or other portions of the surgical article in any number of ways, including printing the alphanumeric identifier on the tag, handle, or other portions of the surgical article.

Similar to the optically-scannable elements as described above, each one of the human-readable elements may be encapsulated in biocompatible plastic coating or housing that is water-impermeable and sterilizable. Each human-readable element may be rigid to increase its service life. In other embodiments, the human-readable element can be flexible to permit the surgical article and the human-readable element to be folded or otherwise shaped in a more preferable manner for use within a patient's body.

The one or more detecting elements may allow an HCP to determine a location of the surgical article within the body of the patient, within an operating room, or both inside the body of the patient and within the operating room. In certain other embodiments, the detecting element may be detectable within the operating room but not within the body of the patient.

The surgical article may include one, two, three, four, or more detecting elements to determine the location of the surgical article. Similar to the counting elements, one or more detecting elements may be incorporated into the tags, the handle, or other portions of the surgical article in any number of ways. For example, the detecting element can be adhered to or encapsulated within the tags, embedded within the handle, or coupled to other portions of the article. In one embodiment, the surgical article can have diametrically opposite sides or corners, and two detecting elements may be coupled to those opposing corners of the article so as to space apart the detecting elements by a maximum distance, and thus reduce the probability of both detecting elements being damaged and rendered undetectable. However, it is contemplated that the detecting elements may be disposed adjacent to one another or coupled to any suitable portion of the article.

The detecting element may be configured to cooperate with at least one remote detector-interrogating antenna (detecting antenna) of a reader, such as a hand-held wand manipulated by the HCP. However, it is contemplated that any suitable antenna, including one integrated within the optical-scanning device can be configured to detect the detecting element.

Each detecting element can be detectable using more than one detection modality. The types of detection modality that can be utilized to detect the detecting elements are not particularly limited, and may include a metal detection modality, a radio detection modality, a magnetic detection modality, an acoustic detection modality, or combinations thereof. It is also contemplated that different types of the same detection modality may be utilized. For example, the system may use two different types of a radio detection modality to detect the detecting elements on the surgical article. In particular, as described in detail below, the detecting element may comprise an electronic article surveillance element (EAS element), a RFID element (RFID element), or any suitable combination thereof. The combinations of different detecting elements, which are responsive to different detection modalities on a single article, permit multiple modes of detection to detect the detecting element and the corresponding surgical article.

Similar to the counting elements, each one of the detecting elements may be encapsulated in biocompatible plastic coating or housing that is water-impermeable and sterilizable. Each detecting element may be rigid to increase its service life. In other embodiments, the tag containing the detecting element can be flexible to permit the surgical article and the tag to be folded or otherwise shaped in a more preferable manner for use within a patient's body.

A wide variety of detecting elements may be commercially available by a number of manufacturers. Certain detecting elements may be configured to provide significant amounts of user accessible memory, sometimes in the form of read-only memory or write-once memory. The amount of memory can vary and determine the size and cost of the integrated circuit of the detecting element. For example, a detecting element produced by TEXAS INSTRUMENTS of Dallas, Tex., under the designation “TAG-IT” may provide 256 bits of user programmable memory in addition to 128 bits of memory reserved for items, such as the unique serial number, version and manufacturing information, and the like. Similarly, a detecting element produced by PHILIPS SEMICONDUCTORS of Eindhoven, Netherlands, under the designation “I-CODE” can provide 384 bits of user memory along with an additional 128 bits reserved for the aforementioned types of information.

Examples of the detecting element can comprise a RFID element detectable by a RFID detecting antenna and an EAS element detectable by an EAS detecting antenna. However, it is contemplated that the surgical article can include any suitable detecting element detectable by any corresponding detecting antenna. As a further example, another embodiment of the RFID detecting element can be detectable by the optical-scanning device, which is configured to scan the optically-scannable element and detect the RFID element.

For one embodiment, each of the detecting elements may comprise a RFID element incorporated into the tags, the handle, or other portions of the surgical article in any number of ways. For example, the RFID element can be adhered to or encapsulated within the tags, embedded within the handle, or coupled to other portions of the article. In one embodiment, the article can have diametrically opposite sides or corners, and two RFID elements may be coupled to those opposing corners of the article.

The RFID element may be encapsulated in biocompatible plastic coating or housing that is water-impermeable and sterilizable. Each RFID element may be rigid to increase its service life. In other embodiments, the tag containing the RFID element can be flexible to permit the surgical article and the tag to be folded or otherwise shaped in a more preferable manner for use within a patient's body.

The detecting element may be used with a multiplex detection system. Continuing with the previous embodiment, the RFID element can include a capacitor and an antenna (not shown), which receives power from the detecting antenna of the reader to charge the capacitor of the RFID element. This capacitor becomes the power source for the operation of the unpowered RFID element. The RFID element can have an integrated circuit, which includes a reading function, a carrier frequency modulating function, and a read-only memory portion with a burned-in code. The integrated circuit and corresponding antenna of the detecting element are encapsulated in an enclosure that is resistant to blood, water, or saline solution. Thus, the RFID element can withstand repeated sterilization and be attached to metal instruments which are sterilized and reused multiple times. Depending on the carrier frequency and the type of RFID element, the RFID element can vary significantly in cost, size, and resistance to shielding by intervening tissue.

In another embodiment, the detecting element may comprise an EAS element that cooperates with the detecting antenna of the reader to detect the location of the surgical article. In contrast to the RFID element, certain embodiments of the EAS element may not be used to uniquely identify the surgical article because these elements may be free from the unique identification information for the surgical article. However, similar to the RFID element, the EAS element may be encapsulated to provide biocompatible contact surfaces, water-resistance and/or sterilizability. For example, the EAS element can be enclosed in glass, polymer or a silicone pot, which can permit the repeated sterilization by various suitable methods using heat, gas, chemicals, or gamma radiation. Furthermore, the EAS elements may be incorporated into the tags, the handle, or other portions of the surgical article in any number of ways. For example, the EAS element can be adhered to or encapsulated within the tags, embedded within the handle, or coupled to other portions of the article. Advantageously, the EAS elements can be made quite as compact, for example, about 11 mm by 2.5 mm. Moreover, the compact construction of the EAS elements can be attached to correspondingly small articles such as hemostats, scalpel handles, and possibly the 4″ by 4″ gauze pads. Additionally, the EAS elements can be relatively inexpensive to manufacture and be less shielded by intervening tissue than the RFID elements. In one embodiment, the article can have diametrically opposite sides or corners, and two EAS elements may be coupled to those opposing corners of the article. The EAS element can generally operate in 500 kHz, but more preferably in the range of from 30 kHz to the range from 100 kHz to 150 kHz. Some embodiments of EAS elements can include: an acousto-magnetic element; an electro-magnetic element; a swept RF element; a LF (LC tank circuit) element; or any combination thereof. The EAS element may utilize a frequency range of from 10 Hz to 10 MHz. The EAS element of a missing or unaccounted-for article can be detected within the patient's body and other locations within the operating room.

While the EAS element only permits detection of the location of the sponge, the RFID element achieves the dual purpose of detecting the location of the surgical article and counting or identifying the surgical article. Thus, certain RFID elements may serve as both detection elements and counting elements. The RFID element cooperates with the detecting antenna of the reader to both detect the location of the surgical article and provide data for determining the unique identification information of the surgical article. Some embodiments of the RFID elements may operate above the 1 MHz range. Exemplary frequencies can include about 13.56 MHz (high frequency), a range from 850 to 950 MHz (ultra-high frequency), or a range of microwave frequencies (i.e., 2.45 to 2.55 GHz). The added bandwidth provided by these RFID elements can increase the probability of detecting and finding the corresponding surgical articles within the interrogation zone and within a short period of time. This will allow a number of surgical articles to be identified and inventoried substantially simultaneously, such as the articles lying on the instrument table, or multiple articles contained within a bodily cavity of the patient. Furthermore, while the RFID elements can be significantly more sophisticated than the EAS elements, the RFID elements can more be expensive to manufacture, have a more bulky construction, and have a greater probability of being shielded from detection by intervening tissue or adjacent metallic articles. Based on the respective advantages and disadvantages of the RFID elements, and the EAS elements, the surgical articles can include one or more EAS elements, one or more RFID elements, or combinations thereof.

To accomplish the dual purpose of being detectable and transmitting the unique identification information, some embodiments of the RFID element can include: the LF Ferrite rod; HF Ferrite rod; HF label element; UHF Ferrite element; UHF label element; and/or combinations thereof. Thus, in certain configurations, one or more of the detecting elements included in the article may be detectable by multiple radio detection modalities. The RFID element can be a bead of ferrite with a coil configured to resonate at a designated frequency. Alternatively, the RFID element can be a flexible thread composed of a single loop wire and capacitor element. The detecting antenna of the reader can locate the RFID element by pulsed emission of a wide-band transmission signal, and the RFID element can resonate with a radiated signal, in response to the wide band transmission, at its own single non-predetermined frequency within the wide band range.

In certain embodiments, the RFID element may utilize a frequency range of from 10 MHz to 1 GHz. Examples of the RFID element include an LF Ferrite rod, HF Ferrite rod, HF label element, UHF Ferrite element, and/or UHF label element. In these embodiments, the RFID element may transmit a signal or wave to a system to provide the unique identification information of the article. The RFID element may convey the same unique identification information provided by the optically-scannable element and the human-readable element as described above.

In order to perform one or more of the functions as described above, each tag may comprise one or more counting elements, such as the optically-scannable element and the human-readable element, and one or more detecting elements, such as the RFID element and the EAS element, or combinations thereof. It is possible that multiple tags, handles, or other portions of the surgical article are selected to have complementary features, such that, in combination on a given article, the two tags have counting elements and detecting elements that can perform all the functions described above. Further still, it is possible that certain functions are redundant across multiple tags as also described above. For example, it is possible that two tags are capable of communicating the same unique identification information for the corresponding surgical article. It is also contemplated that the surgical article may include tags, handles, or other portions of the surgical article having counting/detecting elements that are only capable of collectively performing only one, two, three or four of the functions outlined above.

Referring to FIG. 1A, a plurality of surgical articles may be combined in a package. In the figure, one embodiment of the surgical articles can be a pack of surgical sponges 10 including at least a first surgical sponge 12 and a second surgical sponge 14, which are configured to be counted and detected during a surgical procedure. It is contemplated that the pack can include any number of sponges. In this embodiment, the pack 10 includes a master tag 16, which can be coupled to a ribbon, band, or other packaging holding the sponges together. It should be appreciated that the type of pack or packaging is not particularly limited.

As best shown in FIG. 1B, the master tag 16 comprises a master optically-scannable element 18 that provides the unique identification information for each one of the sponges in the pack 10. The master tag 16 can also include a master human-readable element 20 that provides the unique identification information for each one of the sponges in the pack 10. In other embodiments, the master tag could comprise one or more master detecting elements 21 (FIG. 1A), such as RFID elements including the identification data for all of the sponges in the pack.

The single master tag 16 associated with the pack 10 may be scanned to count-in all the sponges contained in the pack 10. Upon the scanning of this single master tag 16, the optical-scanning device generates multiple article event records, one for each sponge in the pack 10. If the sponges are of the variety for which reconciliation is required, then the article event records are recorded in a reference table to indicate the same. When these article event records are generated, each record contains the unique identification information corresponding with the specific sponge or article with which the record is associated. These sets of unique identification information may be from identification data read from the single master tag on the pack 10 containing the sponges.

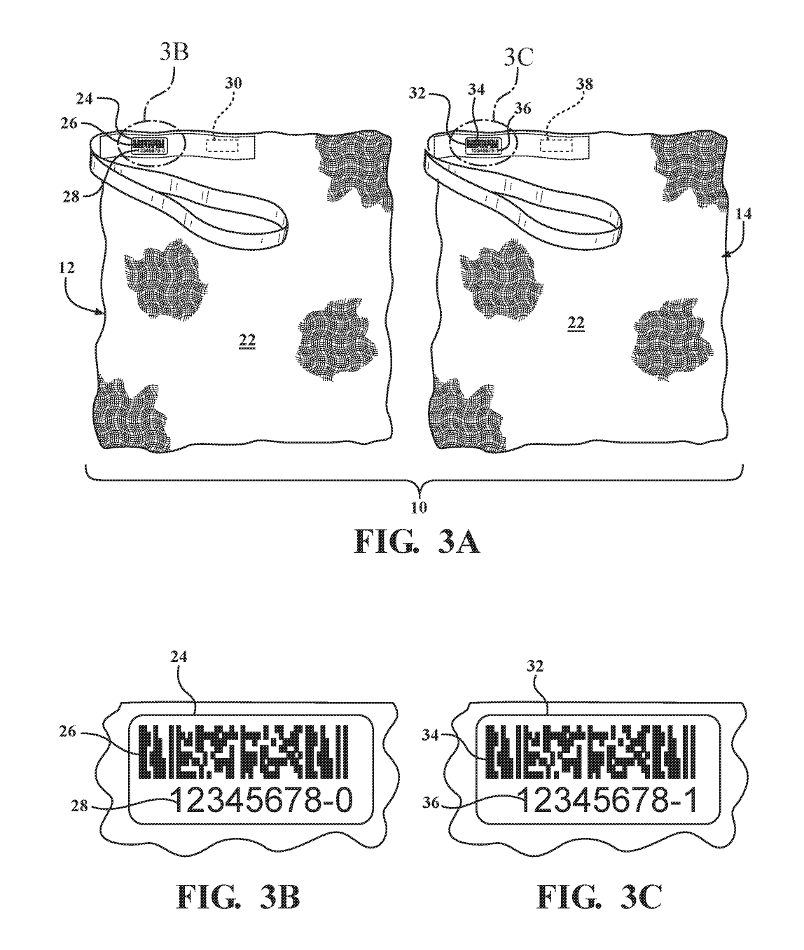

Alternatively, with reference to FIGS. 3A through 3C, the optically-scanning device can generate the unique identification information for all sponges in the pack 10 by scanning the optically-scannable element of any one of the sponges in the pack 10. Thus, based on data in a reference lookup table, the optical-scanning device can generate article event records each with its own set of unique identification information. For example, if the data in the reference lookup table indicates that a serial number XXXX-XXXX-XXXX correlates with a set of unique identification information for a package of five sponges, the scanner will generate five article event records. The article identification field for these records may contain the following format for individual identification numbers XXXX-XXXX-XXXX-0; XXXX-XXXX-XXXX-1; XXXX-XXXX-XXXX-2; XXXX-XXXX-XXXX-3; and XXXX-XXXX-XXXX-4. However, it is contemplated that this field can have various other suitable formats.

In the present embodiment of FIGS. 3A-3C, the first surgical sponge 12 includes an absorbent body 22 and a first tag 24 coupled to the absorbent body 22. The first tag 24 may be a label attached to an outer surface of the absorbent body 22 by an adhesive and thus facilitate with disposing the label within a line of sight of the optical-scanning device. The first tag 24 includes a first optically-scannable element 26, a first human-readable element 28, and a first detecting element 30. The first optically-scannable element 26 and the first human-readable element 28 comprise the first set of unique identification information. Similarly, the second surgical sponge 14 includes an absorbent body 22 and a second tag 32 coupled to the absorbent body 22. The second tag 32 may be a label attached to an outer surface of the absorbent body 22 by an adhesive, and thus making the label visible within a line of sight of the optical-scanning device. The second tag 32 includes a second optically-scannable element 34, a second human-readable element 36, and a second detecting element 38. The second optically-scannable element 34 and the first human-readable element 36 comprise the second set of unique identification information. The first and second sets of unique identification information are different from one another.

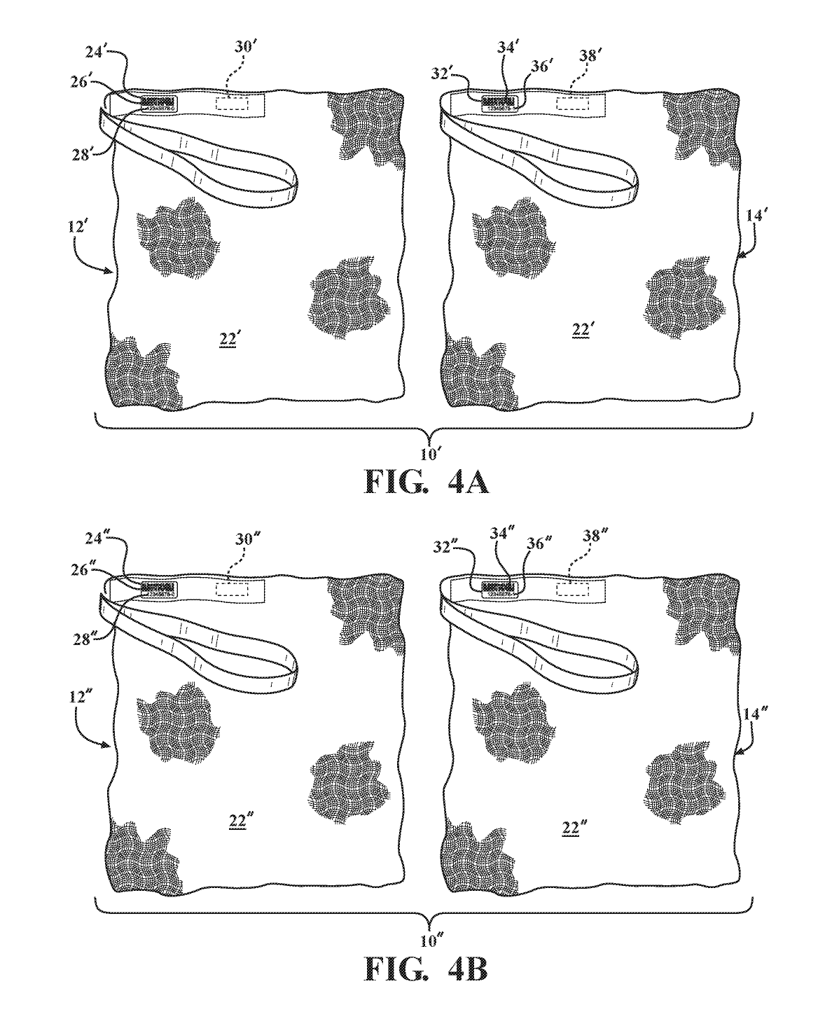

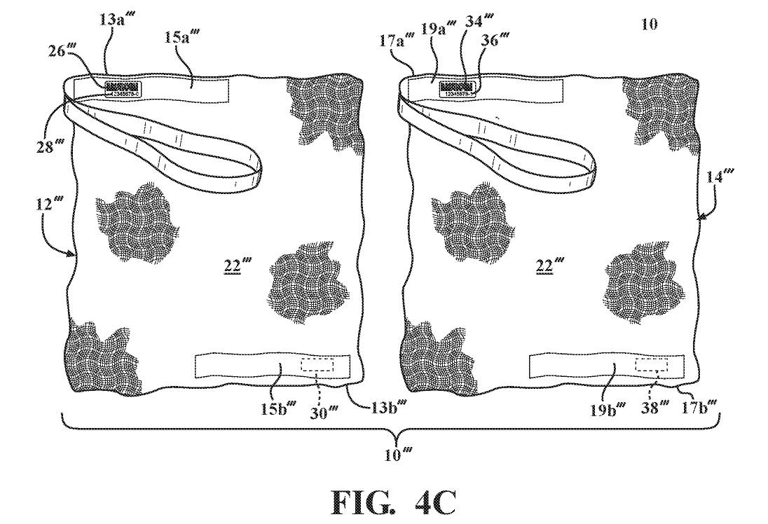

Referring now to FIG. 4A, another exemplary pack of sponges 10′ is similar to the pack of sponges 10 shown in FIG. 3A, and thus the pack of sponges 10′ can include similar components identified by the same reference numbers as followed by a single prime. However, while the first detecting element 30 of FIG. 3A does not include the first set of unique identification information, the first detecting element 30′ of FIG. 4A includes the first set of unique identification information. Moreover, while the second detecting element 38 of FIG. 3A does not include the second set of unique identification information, the second detecting element 38′ of FIG. 4A includes the second set of unique identification information. Both of the first detecting element 30′ and the second detecting element 38′ are detectable within a body of a patient. Some embodiments of the first and second detecting elements 30′, 38′ include a LF ferrite element, HF ferrite element, or combinations thereof

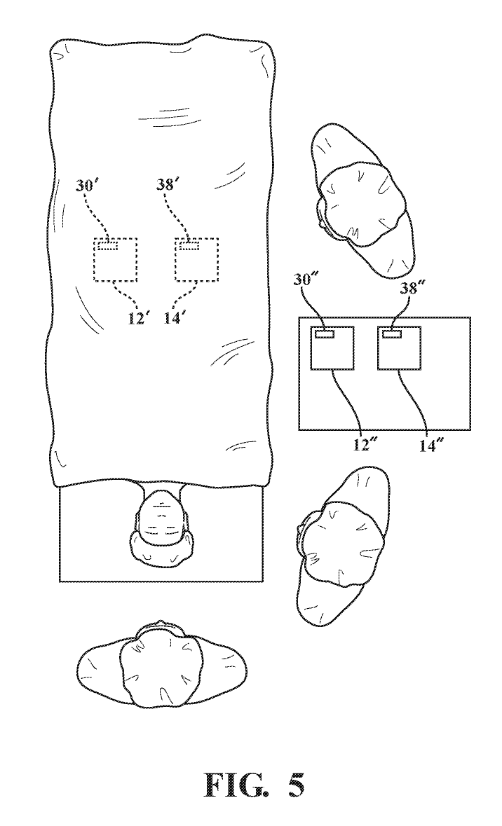

Referring to FIG. 4B, yet another non-limiting exemplary pack of sponges 10″ is similar to the pack of sponges 10′ shown in FIG. 4A, and thus the pack of sponges 10″ can include similar components identified by the same reference numbers as followed by a double prime. However, as shown in FIG. 5, while both of the first and second detecting elements 30′, 38′ of FIG. 4A are detectable within the body of the patient, the first and second detecting elements 30″, 38″ of FIG. 4B are detectable within the operating room but not within the body of the patient. Non-limiting embodiments of the first and second detecting elements 30″, 38″ can include an HF label element, UHF ferrite element, UHF label element, and combinations thereof. Any one of the sponges can include one or more EAS elements, RFID elements, or combinations thereof to permit multiple modalities of detection.

Referring to FIG. 4C, still another embodiment of a pack of sponges 10′″ is similar to the pack of sponges 10 of FIG. 3A, and thus the pack 10′″ includes similar components identified by the same reference numbers followed by a triple prime. The first surgical sponge 12′″ in this form can have more than one tag. For example, the absorbent body 22′″ of the first sponge 12′″ can include two opposite corners 13a′″, 13b′″ and two tags 15a′″, 15b′″ in a corresponding one of the corners 13a′″, 13b′″. One tag 15a′″ can include the first optically-scannable element 26′″ and the first human-readable element 28″, and the other tag 15b′″ can include the first detecting element 30′″. One benefit of a sponge having multiple spaced apart tags and corresponding elements is the decreased risk of damage to all of the elements when, for example, the sponge is damaged and one tag is inadvertently cut. As described above, the first optically-scannable element 26′″ can be a bar code, a data matrix, or combinations thereof. The first optically-scannable element 26′″ and the first human-readable element 28′″ include a first set of unique identification information corresponding with the first surgical sponge 12′″. The first detecting element 30′″ in this embodiment is free from unique identification information. As also described above, embodiments of the first detecting element 30′″ that are free from unique identification information can include an acousto-magnetic element, an electromagnetic element, a swept radio-frequency element, an inductor-capacitor tank circuit element, and combinations thereof. However, other embodiments of the first detecting element 30′″ may have the unique identification information.

The second surgical sponge 14′″ includes an absorbent body 22′″ and more than one tag 19a′″, 19b′″ coupled to the absorbent body 22′″. The combination of tags 19a′″, 19b′″ includes a second optically-scannable element 34″, a second human-readable element 36″, and a second detecting element 38′″. With attention to the embodiment shown in FIG. 4C, the absorbent body 22′″ can include two opposite corners 17a′″, 17b′″ and two tags 19a′″, 19b′″ in a corresponding one of the corners 17a′″, 17b′″. One tag 19a′″ can include the second optically-scannable element 34′″ and the second human-readable element 36″, and the other tag 19b′″ can include the second detecting element 38′″. The second optically-scannable element 34′″ can be a bar code, a data matrix, or combinations thereof. The second optically-scannable element 34′″ and the second human-readable element 36′″ include a second set of unique identification information corresponding with the second surgical sponge 14′″. The second set of unique identification information is different from the first set of unique identification information. One exemplary benefit is that each surgical sponge can be identified, counted, and inventoried within an operating room during the surgical procedure. In this embodiment, the second detecting element 38′″ is free from unique identification information. However, other embodiments of the second detecting element 38′″ may have the unique identification information.

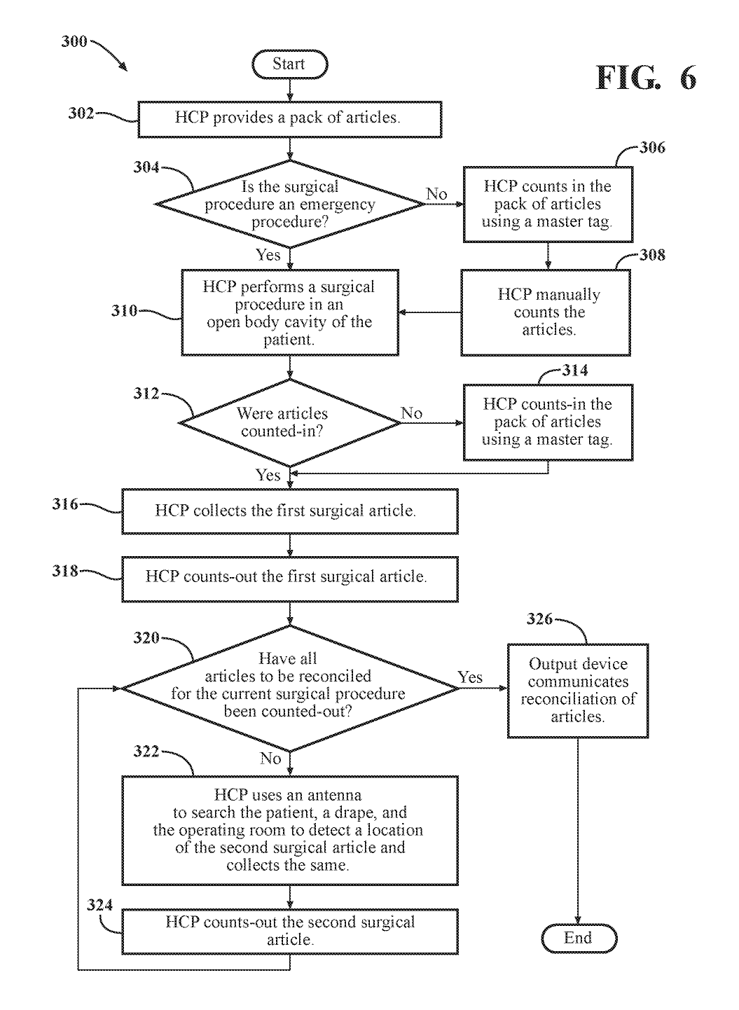

Referring to FIG. 6, a flowchart of a non-limiting exemplary method 300 for managing surgical articles during a procedure that opens one or more bodily cavities is provided. A typical process starts when the article is prepared for introduction into the sterile field. The HCP performing the inventory can make a written record regarding the nature of the article that may be applied to, placed in, or used on the patient. Generally, this written record can provide one basis for determining whether all the recorded articles are accounted for near or at the end of the surgical procedure. The inventory of articles introduced during the procedure is compared to the near post-procedure inventory. In one embodiment, the comparison indicates that there has been a proper accounting of the articles. If there is a discrepancy in the inventory, the medical/surgical team is placed on notice that there must be an accounting for the apparently missing article. In one example, this step can be accomplished by sending a signal to the output device of the user interface sub-assembly to communicate that there must be an accounting for the apparently missing article. This provides the medical/surgical team the opportunity to determine if the article was inadvertently left in the patient prior to closing the patient at the end of the procedure.

At step 302, the method 300 begins with the HCP providing a pack of surgical articles, such as the pack of sponges 10 of FIG. 1A. The pack of sponges 10 includes at least the first surgical sponge 12 and the second surgical sponge 14 as described above (or any of the other surgical articles described above). It is contemplated that the pack of sponges can include any number of sponges in addition to the first and second surgical sponges. Furthermore, the pack may include packaging that retains the plurality of sponges. In certain embodiments, the method may merely include providing the first and second surgical sponges without providing the distinct pack.

The first surgical sponge 12 comprises a first optically-scannable element 26, a first human-readable element 28, and a first detecting element 30. While both of the first optically-scannable element 26 and the first human-readable element 28 comprise a first set of unique identification information, the first detecting element 30 does not include the first set of unique identification. The second surgical sponge 14 comprises a second optically-scannable element 34, a second human-readable element 36, and a second detecting element 38. While both of the second optically-scannable element 34 and the second human-readable element 36 comprise a second set of unique identification information, the second detecting element 38 in this form does not include the second set of unique identification. The first set of unique identification information is different from the second set of unique identification information, such that the first surgical sponge 12 can be distinguished from the second surgical sponge 14 and provide information facilitating use of the sponge. The first and second sets of unique identification information provide serial numbers or other unique identifiers used to distinguish one sponge from another. However, the first and second sets of unique identification information can provide additional information. As one example, the first set of unique identification information can indicate that the first surgical sponge 12 is configured for absorbing bodily fluid in an abdominal region, and the second set of unique identification information can indicate that the second surgical sponge 14 is configured for absorbing bodily fluid within an upper thigh of the patient, thus assisting the HCP with efficiently locating, placing, and removing the sponges from their corresponding locations.

At step 304, the HCP optionally determines whether the surgical procedure is an emergency surgical procedure. If the surgical procedure is an emergency procedure to be performed as soon as possible, the method 300 proceeds immediately to step 310. However, if the surgical procedure is not an emergency procedure, the method 300 proceeds to step 306.

At step 306, the method 300 continues with a counting-in process, which can be used to count-in and identify the pack of sponges 10. In one embodiment, the HCP counts in the pack of sponges 10 by optically scanning one or more counting elements corresponding with the pack of sponges 10. In one example, the counting element can be the master-optically scannable element 18 on the master tag 16, and all sponges in the pack can be counted-in in a single step by using the optical-scanning device to optically scan the bar code or other master optically-scannable element 18 on the master tag 16, which contains the unique identification information of each sponge in the pack 10. In another embodiment, this step can be accomplished by using the detecting antenna to detect the detecting element of the master tag 16, such as the RFID element, which comprises the unique identification information for each sponge.

The optical-scanning device can include a reference lookup table or archive for the sponges 12, 14 to determine or provide the set of unique identification information and count-in each one of the sponges 12, 14. In one embodiment, the HCP can manually input into a controller (not shown) the unique identification information corresponding with the master human-readable element 20 of the master tag 16, which in turn provides the unique identification information data for each one of the sponges 12, 14 in the pack 10. The HCP may utilize the optical-scanning device to create a record of sponges that need to be recovered near the end or after the surgical procedure. In the present embodiment, the HCP can count-in or scan the pack of sponges using the optical-scanning device to indicate which sponges are placed within the patient. On the other hand, the HCP may count-in or scan only the sponges placed in the patient during the surgical procedure. In yet another embodiment, the HCP can count-in the pack of sponges 10 by using the detecting antenna to detect the RFID element or other detecting element 21 on the master tag 16 for the entire pack of sponges 10 or by using the detecting antenna to detect the RFID element or other detecting element on the tag that is attached to only one of the sponges. In still another embodiment, the HCP can count-in the sponges 12, 14 by using both the optical-scanning device and the detecting antenna, when actuating the optical-scanning device for the count-in procedure automatically actuates the detecting antenna to perform the count-in procedure. Put another way, the counting elements and the detecting elements (which have the unique identification information) can be used to perform a redundant count and validate one another's count of the surgical article. If the count accomplished by the counting element of the first modality (such as optical scanning) and the count provided by the counting element of the second modality (such as RFID) having the unique identification information are not equal to one another, a signal may be sent to the output device of the user interface sub-assembly to communicate a counting error and instruct the HCP to correct the error by for example restarting the counting process.

At step 308, the HCP can manually count-in each separate one of the sponges of the pack 10 to provide redundancy to step 306 and improve the reliability of the number of sponges previously counted-in. In particular, the HCP may count in the pack of sponges 10 by using the optical-scanning device to optically scan the optically-scannable element 26, 34 on each separate sponge in the pack 10. Continuing with the previous embodiment, the HCP manually and separately counts-in the first sponge 12 and the second surgical sponge 14 by optically scanning both the first optically-scannable element 26 and the second optically-scannable element 34 of FIG. 1A, which comprise a respective one of the first set of unique identification information and the second set of unique identification information. In another embodiment, the HCP may place the detecting antenna, e.g. a wand, within proximity of the first detecting element 30′ and the second detecting element 38′ of FIG. 3A, which comprise the first set of unique identification information and the second set of unique identification information. The HCP may utilize the optical-scanning device to verify the record of sponges that need to be recovered near the end or after the surgical procedure. Again, the HCP can count-in each one of the sponges and input an instruction to identify the sponges being placed in the patient.

At step 310, the HCP (or another person) performs the surgical procedure and places one or more surgical sponges within one or more open bodily cavities. In the present embodiment, the HCP can use the first surgical sponge 12 in one open bodily cavity and a second surgical sponge 14 in another open bodily cavity. It is contemplated that the sponges or other articles may be tracked and inventoried in surgical procedures that do not involve opening a body cavity or place the articles within an open body cavity.

At step 312, it is determined whether the sponges of the pack 10 were previously counted-in. If the sponges were previously counted-in when, for example, the surgical procedure was not an emergency procedure, then the method proceeds immediately to step 316. However, if the sponges were not previously counted-in when, for example, the surgical procedure was an emergency procedure, the method proceeds to step 314.

At step 314, the HCP counts-in the pack of sponges 10. As one example, the HCP may use the optical-scanning device to optically scan one or more counting elements corresponding with the pack of sponges 10. More specifically, the HCP may use the optical-scanning device to optically scan the master optically-scannable element 18 on the master tag 16, which had been retained after one or more sponges were taken from the pack 10 and used in the surgical procedure. In another embodiment, this step can be accomplished by using the detecting antenna to detect the RFID element (or other detecting element that includes identification information), coupled to the master tag 16.

At step 316, the HCP collects the sponges from the open bodily cavities either during the surgical procedure or after the surgical procedure has been completed and prior to closing the bodily cavities. In one surgical procedure, the HCP may find and collect all of the sponges from the pack 10. However, in another surgical procedure, the HCP may find and collect only a portion of the sponges 12, 14 from the pack 10 because the remaining portion of sponges may be obscured from a direct line of sight, thus preventing the HCP from finding those sponges. In particular, the HCP may collect only the first surgical sponge 12 because it is the only sponge found by the HCP after visually inspecting the body of the patient. Alternatively, the remaining portion of sponges may be intentionally retained in the patient after completion of a current surgical procedure because those sponges are intended for removal at a later time during a later surgical procedure.

At step 318, the HCP counts-out the sponges that were collected from the open bodily cavities. As the surgical procedure is near completion, the sponges are counted-out to provide an accounting or reconciliation of these articles. The optical-scanning device may automatically indicate that the sponge has been counted-out, in response to scanning the optically-scannable element of the sponge. Alternatively, the HCP can use the input device, such as a keyboard, to enter an instruction into the optical-scanning device to indicate that the sponge has been counted-out. A sponge that has been counted-out of a procedure is considered to be a sponge for which there has been a satisfactory final accounting or full reconciliation.

Continuing with the previous embodiment, the HCP can use the optical-scanning device to count-out the first surgical sponge 12 by optically scanning the first optically-scannable element 26 of the first surgical sponge 12. However, if the first optically-scannable element 26 is cut, sullied, or otherwise rendered inoperable, the HCP can manually input the first human-readable element 28 to provide the first set of unique identification information of the first surgical sponge 12. In another embodiment, the first surgical sponge 12′ of FIG. 4A can be counted-out by detecting the first RFID detecting element 30 to provide the data for determining the first set of unique identification. In yet another embodiment, the first surgical sponge 12″ of FIG. 4B can be counted-out by detecting the first EAS detecting element 30″ and obtaining data for determining the first set of unique identification of the first detecting element 30″. In still another embodiment that utilizes both counting elements and detecting elements, the HCP can use the optical-scanning device to count-out the first surgical sponge 12 by optically scanning the first optically-scannable element 26 of the first surgical sponge 12, which in turn actuates the detecting antenna to count-out the first surgical sponge 12. Put another way, the counting elements and the detecting elements that have the unique identification information can be used to perform a redundant count and validate one another's count of the surgical article. If the count accomplished by the counting element and the count provided by the detecting element having the unique identification information are not equal to one another, a signal may be sent to the output device of the user interface sub-assembly to communicate a counting error and instruct the HCP to correct the error by for example restarting the counting process.

At step 320, it is determined whether all sponges to be reconciled during the current surgical procedure have been counted-out. In one embodiment, based on the records of the counted-in articles, the processor can determine the number of each type of counted-in article for which reconciliation is required and compare the same with the number of those articles that have been counted-out. The difference between these two quantities yields a preliminary number of sponges for which reconciliation may be required. After some procedures, the HCP may intentionally leave some sponges in the patient for removal at a prescribed time during a later surgical procedure, thus reducing the final number of sponges to be reconciled for the current surgical procedure. Specifically, to achieve full reconciliation for the current surgical procedure, the HCP can acknowledge to the system that some sponges are being intentionally left in the patient upon completion of the current surgical procedure, such that the quantity and identity of those sponges can be confirmed during the later surgical procedure. Thus, the processor may determine the final number of sponges to be reconciled during the current surgical procedure by reducing the preliminary number of sponges to be reconciled during the current procedure by the quantity of sponges to be removed at a later time during the later surgical procedure. If the final number is equal to zero, the processor determines that all sponges to be reconciled for the current surgical procedure have been counted-out and transmits a signal to the output device of the user interface sub-assembly to communicate the same. More specifically, in this embodiment, the processor can transmit a signal to the touch screen display, which in turn displays a message that all types of sponges for which reconciliation is required during the current surgical procedure have been reconciled, and the method proceeds to step 326. However, if the processor determines that the final number is greater than zero, the processor can transmit a signal to output device, such as the touch screen display, which in turn displays a warning or other message indicating with specificity the types of sponges for which reconciliation is still required during the current surgical procedure, and the method proceeds to step 322.

At step 322, the HCP detects the locations of the remaining uncollected and unreconciled sponges that require reconciliation for the current surgical procedure, and the HCP collects the same. The articles for which there has not yet been a proper accounting are considered to be unreconciled. In this embodiment, the only remaining uncollected sponge is the second surgical sponge 14. The HCP may bring the detecting antenna in the proximity of areas where the uncollected sponge(s) may be located. The HCP may detect the location of the second detecting element 38 for the second surgical sponge 14 in the bodily cavity of the patient, on the patient outside of the bodily cavity, on a surgical drape, or in other locations within the operating room. Subsequent to finding the second surgical sponge 14, the HCP collects the same.

At step 324, the HCP scans or counts-out the collected and unreconciled sponges. In this embodiment, the HCP counts-out the second surgical sponge 14 by optically scanning the second optically-scannable element 34 of the second surgical sponge 14. However, if the second optically-scannable element 34 is cut, sullied, or otherwise rendered inoperable, the HCP can manually input the second human-readable element 36 to provide the second set of unique identification information of the second surgical sponge 14. In another embodiment, the second surgical sponge 14′ of FIG. 4A can be counted-out by using the second detecting element 38′ because the second detecting element 38′ is an RFID element containing unique identification data that can be used to determine the second set of unique identification. Similarly, the second surgical sponge 14″ of FIG. 4B can be counted-out by scanning the first detecting element 30″ because the first detecting element 30″ is an RFID element containing unique identification data that can be used to determine the second set of unique identification of the second detecting element 38″. As a result of scanning the sponge, the processor may update the table of reconcilable articles to indicate that the sponges that were previously counted-in have been counted-out and thus reconciled.

In another embodiment, the optical-scanning device can have a processor that determines if a record has been generated in the table for the specific type of article. For example, if the article to be reconciled is a 20 cm by 20 cm sponge, the processor determines whether or not the table includes a record for this type of sponge. If the evaluation tests positive for the particular type of article, the number of remaining articles to be collected and reconciled is decreased by one. If the evaluation tests negative, the processor can send a signal to the output device of the user interface sub-assembly, which in turn communicates that this type of article was not previously scanned or counted-in. Continuing with the previous embodiment, the output device may be a touch screen display presenting a warning or message that this type of sponge was not previously scanned or counted-in.

The method then returns to step 320, and if there has not been an accounting or reconciliation of all the sponges to be collected during the current surgical procedure, the processor transmits a signal to the output device of the user interface sub-assembly to communicate that all sponges have not been reconciled for the current surgical procedure. In particular, the touch screen display may present a warning or a message to the HCP to indicate that investigation of the area for missing sponges should be continued and the status of the unaccounted sponges should be identified prior to concluding the procedure and closing the bodily cavity. If the processor determines that all sponges have been reconciled for the current surgical procedure, the method proceeds to step 326.

At step 326, the processor sends a signal to the output device of the user interface sub-assembly to communicate that the sponges to be reconciled during the current surgical procedure have been reconciled. The output device in one form is the touch screen display, which may display a message indicating that reconciliation of the sponges during the current surgical procedure has been completed. Furthermore, the processor can also generate a procedure event record to update the EMR to indicate a successful article reconciliation for the current surgical procedure and provide information directed to sponges that must be collected at a later time.

The invention has been described in an illustrative manner and it is to be understood that the terminology which has been used is intended to be in the nature of words of description rather than of limitation. Many modifications and variations of the present invention are possible in light of the above teachings and the invention may be practiced otherwise than as specifically described.

Embodiments of the disclosure can be described with reference to the following numbered clauses, with specific features laid out in the dependent clauses:

I. A plurality of surgical sponges comprising:

a first surgical sponge including a first optically-scannable element, a first human-readable element, and a first detecting element, both of the first optically-scannable element and the first human-readable element including a first set of unique identification information; and

a second surgical sponge including a second optically-scannable element, a second human-readable element, and a second detecting element, both of the second optically-scannable element and the second human-readable element including a second set of unique identification information,

wherein the first set of unique identification information is different from the second set of unique identification information;

wherein both of the first detecting element and the second detecting element are free from unique identification information;

wherein the first detecting element is selected from the group consisting of an acousto-magnetic element, an electromagnetic element, a swept radio-frequency element, an inductor-capacitor tank circuit element, and combinations thereof.

II. A plurality of surgical sponges comprising:

a first surgical sponge including a first optically-scannable element, a first human-readable element, and a first detecting element, each one of the first optically-scannable element, the first human-readable element, and the first detecting element including a first set of unique identification information; and

a second surgical sponge including a second optically-scannable element, a second human-readable element, and a second detecting element, each one of the second optically-scannable element, the second human-readable element, and the second detecting element including a second set of unique identification information,

wherein the first set of unique identification information is different from the second set of unique identification information, and

wherein both of the first detecting element and the second detecting element are detectable within a body of a patient.

III. The plurality of surgical sponges of clause II, wherein the first surgical sponge includes an absorbent body and a tag coupled to the absorbent body, the first tag includes the first optically-scannable-element and the first human-readable element.

IV. The plurality of surgical sponges of any one of clauses II or III, wherein the first detecting element is selected from the group consisting of a low-frequency ferrite detectable element, a high-frequency ferrite detectable element, and combinations thereof.

V. A plurality of surgical sponges comprising:

a first surgical sponge including a first optically-scannable element, a first human-readable element, and a first detecting element, all of the first optically-scannable element, the first human-readable element, and the first detecting element including a first set of unique identification information; and

a second surgical sponge including a second optically-scannable element, a second human-readable element, and a second detecting element, all of the second-optically scannable element, the second human-readable element, and the second detecting element including a second set of unique identification information,

wherein the first set of unique identification information is different from the second set of unique identification information, and

wherein the first detecting element and the second detecting element are both not detectable within a body of a patient.

VI. The plurality of surgical sponges of clause V, wherein the first remotely-detectable element is selected from the group consisting of a high-frequency label element, an ultra high-frequency ferrite element, an ultra high-frequency label element, and combinations thereof.

VII. The plurality of surgical sponges of any one of clauses V or VI, wherein the first surgical sponge includes an absorbent body and a tag coupled to the absorbent body, the tag including the first optically-scannable-element and the first human-readable element.

1. A method for managing surgical sponges in an operating room during a surgical procedure, the method comprising:
providing a first surgical sponge and a second surgical sponge, the first surgical sponge including a first optically-scannable element, a first human-readable element, and a first detecting element, both of the first optically-scannable element and the first human-readable element including a first set of unique identification information, the second surgical sponge including a second optically-scannable element, a second human-readable element, and a second detecting element, both of the second optically-scannable element and the second human-readable element including a second set of unique identification information, wherein the first set of unique identification information is different from the second set of unique identification information;
counting-in the first and second surgical sponges by optically-scanning;
collecting the first surgical sponge;
counting-out the first surgical sponge;
detecting a location of the second surgical sponge using the second detecting element after collecting the first surgical sponge;
collecting the second surgical sponge after the step of detecting the location of the second surgical sponge; and
counting-out the second surgical sponge.

2. The method of claim 1, wherein the step of providing the first surgical sponge and the second surgical sponge includes providing a pack of surgical sponges.

3. The method of claim 2, wherein the step of counting-in the pack of surgical sponges by optically-scanning includes optically scanning a master tag corresponding with the pack of sponges with the master tag providing the first and second sets of unique identification information.

4. The method of claim 1, wherein the step of counting-in the first and second surgical sponges by optically-scanning includes optically scanning a first tag corresponding with the first surgical sponge and a second tag corresponding with the second surgical sponge.

5. The method of claim 1, wherein the step of counting-out the first surgical sponge includes optically-scanning the first optically-scannable element of the first surgical sponge.

6. The method of claim 1, wherein the step of counting-out the first surgical sponge includes manually inputting the first human-readable element of the first surgical sponge.

7. The method of claim 1, wherein the step of counting-out the second surgical sponge includes optically-scanning the second optically-scannable element of the second surgical sponge.

8. The method of claim 1, wherein the step of counting-out the second surgical sponge includes manually inputting the second human-readable element of the second surgical sponge.

9. The method of claim 1, wherein the second detecting element includes the second set of unique identification information, and wherein the step of counting-out the second surgical sponge includes counting-out the second surgical sponge using the second set of unique identification information of the second detecting element.

10. The method of claim 1, wherein the step of detecting the location of the second surgical sponge includes detecting the second detecting element within a body of a patient.

11. The method of claim 1, wherein the step of detecting the location of the second surgical sponge includes detecting the second detecting element within an operating room but outside of a body of a patient.

12. The method of claim 1, wherein the step of counting-out the first surgical sponge includes determining the first set of unique identification information.

13. A plurality of surgical sponges comprising:
a first surgical sponge including a first optically-scannable element, a first human-readable element, and a first detecting element, both of the first optically-scannable element and the first human-readable element including a first set of unique identification information; and
a second surgical sponge including a second optically-scannable element, a second human-readable element, and a second detecting element, both of the second optically-scannable element and the second human-readable element including a second set of unique identification information,
wherein the first set of unique identification information is different from the second set of unique identification information; and
wherein both of the first detecting element and the second detecting element are free from unique identification information.

14. The plurality of sponges of claim 13, wherein the first surgical sponge includes an absorbent body and a tag coupled to the absorbent body, the first tag includes the first optically-scannable element and the first human-readable element.

15. The plurality of surgical sponges of claim 13, wherein the first optically-scannable element is selected from the group consisting of a bar code, a data matrix, and combinations thereof.

16. A plurality of surgical sponges comprising:
a first surgical sponge including a first optically-scannable element, a first human-readable element, and a first detecting element, all of the first optically-scannable element, the first human-readable element, and the first detecting element including a first set of unique identification information; and
a second surgical sponge including a second optically-scannable element, a second human-readable element, and a second detecting element, all of the second-optically scannable element, the second human-readable element, and the second detecting element including a second set of unique identification information,
wherein the first set of unique identification information is different from the second set of unique identification information, and
wherein the first detecting element and the second detecting element are both not detectable within a body of a patient.

17. The plurality of surgical sponges of claim 16, wherein the first remotely-detectable element is selected from the group consisting of a high-frequency label element, an ultra high-frequency ferrite element, an ultra high-frequency label element, and combinations thereof.

18. The plurality of surgical sponges of claim 16, wherein the first surgical sponge includes an absorbent body and a tag coupled to the absorbent body, the tag including the first optically-scannable-element and the first human-readable element.
